(19) United States
(12) Patent Application Publication
(10) Pub. No.: US 2009/0326545 A1
(43) Pub. Date: Dec. 31, 2009

(54) Systems and methods for inserting a bone anchor without a pilot hole

(75) Inventor: Cory R. Schaffhausen, Salt Lake City, UT (US)

Correspondence Address: MINTZ LEVIN COHN FERRIS GLOVSKY & POPEO, ONE FINANCIAL CENTER, BOSTON, MA, 02111, US

(73) Assignee: Amedica Corporation, Salt Lake City, UT (US)

(21) Appl. No.: 12/215,482

(22) Filed: Jun. 26, 2008

(51) Int. Cl. A61B 17/58; B25B 13/48

(52) U.S. Cl. 606/104; 81/436

(57) ABSTRACT

Bone anchor insertion assemblies and methods are provided for inserting a bone anchor (e.g., bone screw) into bone without having to create a pilot hole. In some embodiments, the assembly includes a bone anchor, a housing member, and a rod. The housing member, which is coupled to the rod, is movable between proximally and distally advanced positions relative to the rod. In the proximal position, the bone anchor can be coupled to the rod. In the distal position, a tip of the bone anchor is generally aligned with a distal end of the housing member. The bone anchor is advanced into the bone under guidance of the housing member, where the tip of the bone anchor serves as a bone awl. Once the bone anchor is seated within the bone, the housing member can be returned to the proximal position to improve visibility of the surgical procedure.

### FIELD OF THE INVENTION

Embodiments of the present invention relate to bone anchor insertion assemblies and tools that can be used to insert a bone anchor (e.g., bone screw) into a bone without having to create a pilot hole. Other embodiments of the present invention relate to methods for inserting a bone anchor into a bone without having to create a pilot hole.

### BACKGROUND OF THE INVENTION

The spine is a flexible, multi-segmented column that supports upright posture in a human while providing mobility to the axial skeleton. The spine encases and protects vital neural elements while providing structural support for the body by transmitting the weight of the body through the pelvis to the lower extremities. The cervical spine exhibits a wide range of motion due to the orientation of its facets and the lack of supporting structures. The thoracic and lumbar regions of the spine also have significant ranges of motion.

The spine is made up primarily of bone and intervertebral discs, which are surrounded by supporting ligaments, muscle, fascia, blood vessels, nerves, and skin. These elements are subject to a variety of pathological disturbances: inflammation, trauma, neoplasm, congenital anomalies, disease, etc. Trauma to the spine can play a large role in the etiology of neck and low back pain. For example, trauma frequently results in damage at the upper end of the lumbar spine, where the mobile lumbar segments join the less mobile dorsal spine. Excessive forces on the spine not only produce life-threatening traumatic injuries, but may contribute to an increased rate of degenerative change.

The cervical region of the spine includes the seven most superior vertebrae of the spine, which begin at the base of the skull and end at the upper torso. Because the neck has a wide range of motion and is the main support for the head, the neck is extremely vulnerable to injury and degeneration.

Spinal fixation is a common method of treating spinal disorders, fractures, and degeneration. One common device used for spinal fixation is the bone fixation plate, which is typically used in conjunction with a graft device placed between the vertebral bodies. A typical bone fixation plate includes a relatively flat, rectangular plate having a plurality of apertures formed therein. A corresponding plurality of bone screws may be provided to secure the bone fixation plate to the vertebrae of the spine.

Many of the screws used to secure spinal fixation plates are considered to be “self-drilling and self-tapping” screws, which generally have cutting flutes. However, these screws normally are not used in practice without a pilot hole. One reason is that if the threads of these self tapping screws do not engage the bone early enough, the self drilling cutting flutes can enlarge the hole beyond what is desired. Also, without a pilot hole, a surgeon must center the screw tip in the screw bore by sight. If the screw is placed off center, the threads may interfere with or damage a locking mechanism of the bone plate or the screw head may not sit flush inside the screw bore.

As a result many surgeons create a pilot hole using a bone awl. Surgical bone awls are known and include ones with a sleeve over the awl, such as in U.S. Pat. No. 5,957,946 to Shuler et al. The sleeve of the bone awl may be seated in the center of the screw bore of a spinal fixation plate to center the awl. However, the present inventor notes that the use of a separate bone awl to create a pilot hole adds extra steps, complexity and time to the surgical procedure.

Another system used to insert and align a bone screw is the Vectra™ Anterior Cervical Plate System (Synthes Spine). In the aforementioned system, a guide post is inserted into the screw bore of a cervical plate. A drill is inserted into the barrel of the guide post, a pilot hole is drilled, and the drill is removed. A screw with a driver is then inserted into the barrel of the guide post, and the driver is used to advance the screw into the bone. However, the present inventor notes that the use of a separate drill to create a pilot hole adds extra steps, complexity and time to the surgical procedure. Further, throughout the advancement of the screw until or just before the screw head is seated within the bone plate, the guide post remains within the screw bore of the cervical plate, thus limiting the ability of a surgeon to visually monitor the procedure.

In view of the foregoing, it would be desirable to provide improved systems, methods and tools for insertion of a bone screw into a bone.

### SUMMARY OF THE INVENTION

Some embodiments of the present invention relate to bone screw insertion assemblies, tools, and methods for inserting a bone screw into a bone without having to create a pilot hole.

In one aspect, a bone anchor (e.g., bone screw) insertion assembly is provided that includes a housing member and a rod. The housing member (e.g., elongated sleeve) has a distal end and a proximal end, where the distal end of the housing member is configured to receive a bone anchor having a proximal end and a tip at its distal end. In a distally advanced position of the housing member, the tip of the bone anchor is generally aligned with the distal end of the housing member. Upon advancement of the bone anchor under guidance of the housing member, the tip of the bone anchor itself (e.g., having a length of about 2 mm) can serve as a bone awl. The rest of the bone anchor is at least partially positioned within the housing member. The rod, which is coupled to the housing member (e.g., configured for slidable engagement), is capable of detachably coupling to the proximal end of the bone anchor, where advancement of the rod (e.g., from impact or rotation) causes the bone anchor to advance into bone.

In some embodiments, the tip of the bone anchor may slightly protrude (e.g., about 1-2 mm) from the distal end of the housing member in the distally advanced position of the housing member. In some embodiments, the tip of the bone screw may be generally flush with the distal end of the housing member in the distally advanced position of the housing member. In still other embodiments, the tip of the bone screw may be slightly recessed (e.g., about 1-2 mm) from the distal end of the housing member in the distally advanced position of the housing member.

In some embodiments, the rod and the housing member may share a longitudinal axis along which the rod and housing member can move relative to each other, for example, to permit loading and unloading of the bone anchor within the housing member and to allow the bone anchor to be visible to a surgeon as it is advanced into bone. The distal end of the housing member may be configured to be seated within a bore of a bone fixation plate and secured in place by, for example, a press-fit. Advancement of a handle rigidly coupled (e.g., by welding) to a proximal end of the rod may cause the bone screw to advance into bone.

In some embodiments, the distal end of the housing member may include a flexible portion (e.g., one or more flexible fingers) capable of flexing in a direction away from a longitudinal axis of the housing member to allow the head of the bone anchor to pass through the distal end and into and out of the housing member.

In some embodiments, the rod of the bone anchor insertion assembly includes a tube traversing longitudinally through at least a portion of the housing member and a shaft positioned at least partially within the tube. In some embodiments, the shaft is capable of detachably coupling to the head of the bone anchor. For example, the shaft may be threaded at its distal end for mating with a threaded bore within the head of the bone anchor. The proximal end of the shaft may be coupled (e.g., welded, press-fit, or threaded) to a knob, which can be rotated to thread and unthread the distal end of the shaft into and out of the threaded bore.

In some embodiments, the bone anchor insertion assembly includes a mechanism for facilitating positioning and/or movement of the housing member relative to the rod. For example, in some embodiments, the housing member may include a ball detent that is configured for receipt within a complimentary recess in the rod. When the housing member is advanced distally relative to the rod, the ball detent may be seated within the complimentary recess thus fixing a relative position of the housing and the rod. In this position, the housing member may serve as a guide for insertion of the bone anchor through a bone fixation plate and into bone.

In some embodiments, the housing member and the rod may be configured to transition from this first position to a second position, for example, in which the surgeon can view the bone anchor during the surgical procedure. For example, the housing member may be configured for movement (e.g., slidable engagement) relative to the rod in a distal-to-proximal direction, where such movement may be achieved automatically or as a result of manual force. In some embodiments, a spring is provided that compresses when the housing member is advanced distally into the first position. Following advancement of the bone anchor a predetermined distance (e.g., about 2 mm) into the bone, the bone anchor insertion assembly may transition to the second position. For example, the ball detent of the housing member may enter a sliding channel formed within the rod and, under an expansion force of the spring, the housing member may move in the distal-to-proximal direction as the ball detent traverses the sliding channel in the distal-to-proximal direction.

In some embodiments, an indicator may be provided within the bone anchor insertion assembly that indicates the position of the rod, and the bone screw coupled to its distal end, relative to the housing member. For example, a visual indicator or guide pin may be provided on the rod that is viewable through or within a slot formed in the housing member. The indicator may traverse the slot formed within the housing member in response to relative movement between the housing member and the rod.

In some embodiments, the bone anchor is a bone screw (e.g., having a length of 12-16 mm) having a screw head, a threaded shaft, and a tip at its distal end. The tip of the bone screw (e.g., having a length of about 1-2 mm) may form a sharp point capable of creating a hole in a bone surface. In some embodiments, the diameter of the screw head is larger than the diameter of the threads on the threaded shaft. In some embodiments, the threaded shaft is tapered, for example, with the threads tapering to a diameter of near zero upon reaching the tip.

In still another aspect, methods for inserting a bone anchor (e.g., bone screw) into a bone are provided. A bone anchor having a head and a tip at its distal end may be positioned within a housing member such that the tip of the bone anchor is generally aligned with a distal end of the housing member. The housing member may be coupled to a rod having a distal end, which is coupled to the head of the bone anchor. The tip of the bone anchor may be advanced into bone (e.g., about 1-2 mm) under guidance of the housing member and as a result of advancement of the rod, for example, by impact and/or rotation. The housing member may be retracted relative to the rod either automatically or upon application of manual force. The rod may be further advanced (e.g., impacted and/or rotated), which may cause the bone anchor to advance further into the bone. When the bone anchor is fully advanced into the bone, the head of the bone anchor may be uncoupled from the rod.

In some embodiments, positioning the bone anchor within the housing member includes flexing a distal end of the housing member to permit passage of the head of the bone anchor into the housing member.

In some embodiments, advancing the tip of the bone anchor into the bone includes seating the distal end of the housing member into a bore of a bone fixation plate (e.g., a cervical bone plate). For example, the bore of the bone fixation plate may have the same or similar diameter or sizing as the distal end of the housing member. In some embodiments, advancing the tip of the bone anchor into the bone includes advancing the tip of the bone anchor into bone without creating a pilot hole in the bone.

It is contemplated that whenever appropriate, any embodiment of the present invention can be combined with one or more other embodiments of the present invention, even though the embodiments are described under different aspects of the present invention.

### BRIEF DESCRIPTION OF THE DRAWINGS

For a better understanding of the present invention, including the various objects and advantages thereof, reference is made to the following detailed description, taken in conjunction with the accompanying illustrative drawings, in which like reference characters refer to like parts throughout, and in which:

FIG. 1 is a side view of a bone anchor insertion assembly, which includes a housing member, a rod, a handle, and a bone anchor (e.g., bone screw), according to some embodiments of the present invention;

FIG. 2 is a cross-sectional view of a distal portion of the rod of FIG. 1;

FIG. 3 is a perspective view of another embodiment of a bone screw and a rod according to the present invention;

FIG. 4 is a cross-sectional view of the handle and a proximal portion of the rod of FIG. 1;

FIG. 5A is a cross-sectional, perspective view of a distal portion of the bone anchor insertion assembly of FIG. 1, in which the housing member is in a proximal position relative to the rod and the rod is ready for engagement with a bone anchor;

FIG. 5B is a cross-sectional, perspective view of a distal portion of the bone anchor insertion assembly of FIG. 1, in which the housing member is in a distal position relative to the rod, with the bone anchor loaded within the housing member and coupled to the rod and with the tip of the bone anchor generally aligned with a distal end of the housing member;

FIG. 6A is a, perspective view of part of the bone anchor insertion assembly of FIG. 1, in which the housing member is in a proximal position relative to the rod and in which an indicator is provided that indicates the position of the rod and the bone anchor relative to the housing member according to some embodiments of the present invention;

FIG. 6B is a perspective view of part of the bone anchor insertion assembly of FIG. 6A, in which with the housing member is in a distal position relative to the rod;

FIG. 7 is a perspective view of part of the bone anchor insertion assembly of FIG. 1 showing the features for facilitating positioning and movement of the housing member relative to the rod, according to some embodiments of the present invention;

FIG. 8 is a perspective view of another embodiment of a housing member according to the present invention;

FIG. 9 is a side view of the housing member of FIG. 8 incorporated within a bone anchor insertion assembly according to some embodiments of the present invention;

FIG. 10A is a side view of a distal portion of another bone anchor insertion assembly according to some embodiments of the present invention, in which a housing member is provided that is configured for slidable engagement with a rod; and

FIG. 10B is a perspective view of the bone anchor insertion assembly of FIG. 10A.

### DETAILED DESCRIPTION OF THE INVENTION

Embodiments of the present invention relate to assemblies and methods for inserting a bone anchor (e.g., bone screw) into bone. Advantageously, the assemblies and methods described herein can alleviate the need to create a pilot hole in the bone, thus allowing for a reduction in the complexity of and time required for the surgical procedure.

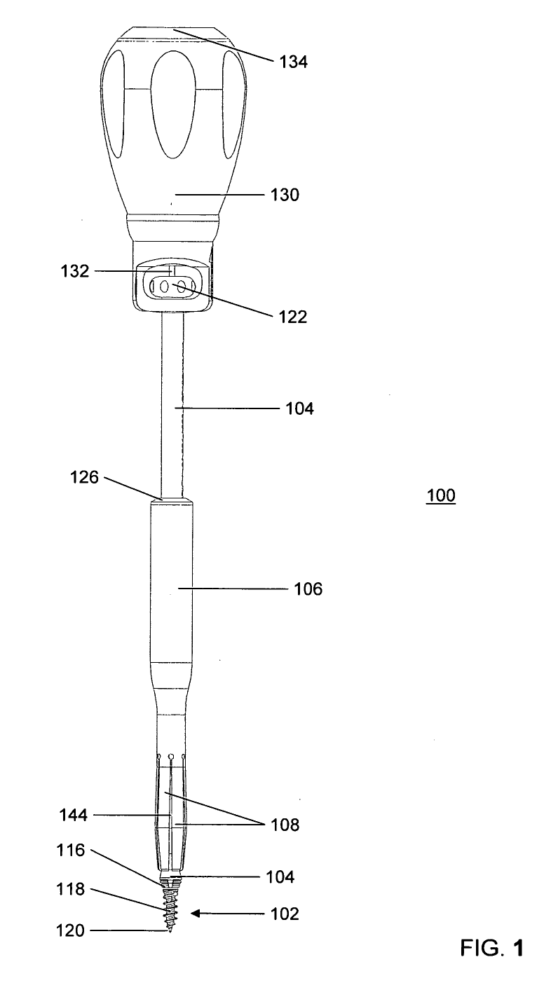

FIG. 1 is a side view of a bone anchor insertion assembly 100 according to some embodiments of the present invention. Assembly 100 includes bone anchor 102, rod 104 and housing member 106. In some embodiments, housing member 106 is a sleeve, for example, a sleeve (e.g., elongated sleeve) with a circular or oval circumference. At the distal or screw receiving end of housing member 106 is a flexible portion capable of flexing in a direction away from a longitudinal axis of housing member 106. In some embodiments, the flexible portion of housing member 106 includes one or more (e.g., four, five or six) flexible members, for example, flexible fingers 108.

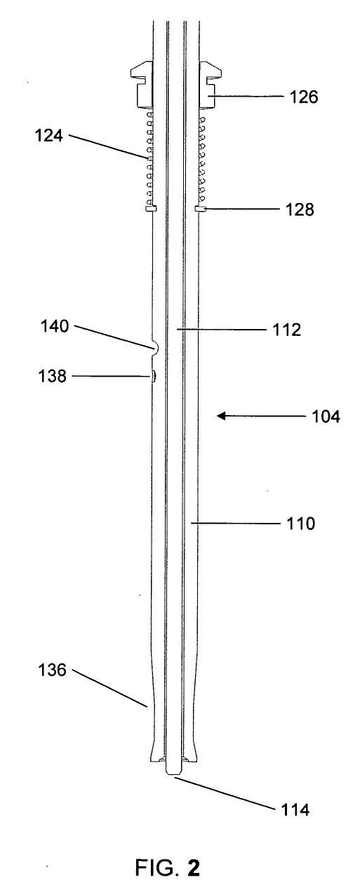

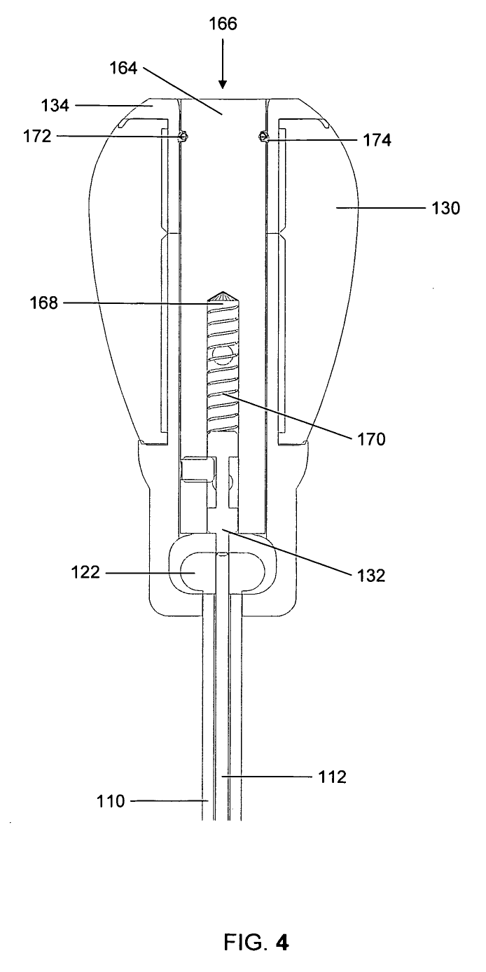

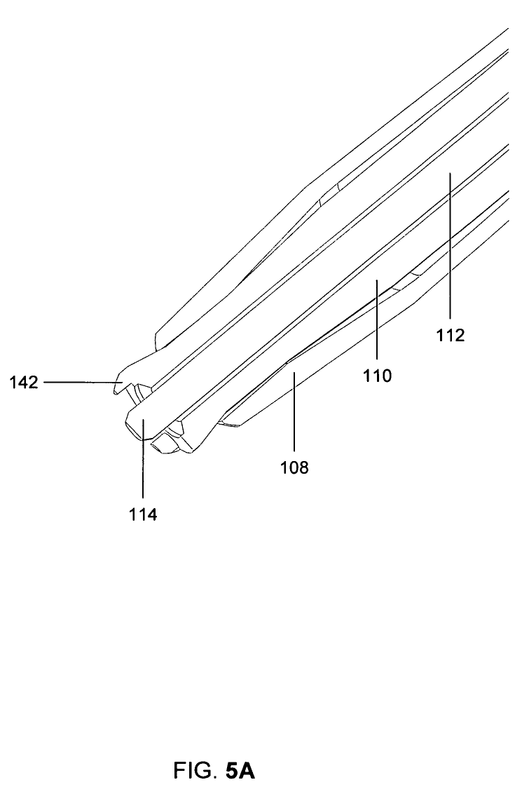

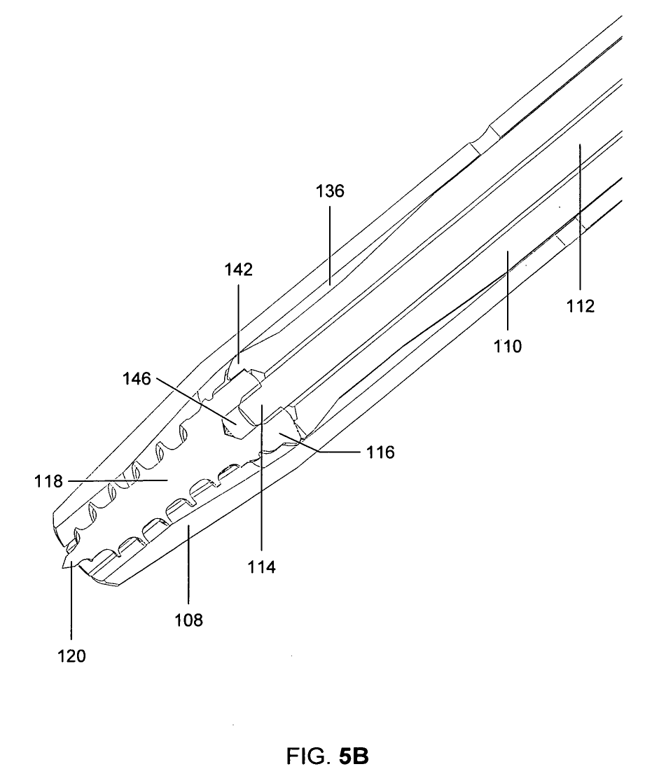

FIG. 2 is an enlarged, cross-sectional view of the distal portion of rod 104 of the embodiment of FIG. 1. With reference to FIGS. 1 and 2, rod 104 runs longitudinally through at least a portion of housing member 106, which can translate axially along rod 104. Rod 104 is configured for detachable coupling to anchor 102. For example, in some embodiments, bone anchor 102 may be a bone screw (FIG. 1) that includes head 116, threaded shaft 118, and tip 120 distal from head 116. The diameter of head 116 may be larger than the diameter of shaft 118. The threads on shaft 118 may taper down to a minor diameter of near zero upon reaching tip 120. In some embodiments, rod 104 includes a tube 110 and a shaft 112 running through tube 110. Referring to FIGS. 2 and 5B, shaft 112 may have a threaded end 114, which can be screwed into a threaded bore 146 in screw head 116 of screw 102 by rotating knob 122 (FIGS. 1 and 4) attached (e.g., by welding or threaded engagement) to the proximal end of shaft 112. In other embodiments, screw head 116 may contain a socket and shaft 112 may have a tapered end with a cross-sectional area that is complementary to the cross-sectional area of the socket. An interference fit may be formed upon inserting the tapered end of shaft 112 into the socket. Detachably coupling bone anchor 102 to shaft 112 may prevent anchor 102 from inadvertently disconnecting from assembly 100 during surgery. Alternatively or additionally, the coupling between bone anchor 102 and shaft 112 may be utilized to advance anchor 102 into bone upon further rotation of knob 122.

In some embodiments, tube 110 may be configured for detachably coupling to bone anchor 102. For example, referring to FIGS. 1, 5A, and 6A, anchor head 116 may include multiple (e.g., three equidistant) recesses 156 positioned around the perimeter of screw head 116 for receiving prongs 142 at a distal end of tube 110. The coupling between bone anchor 102 and tube 110 may be used to advance anchor 102 into bone, by rotating handle 130 which is attached (e.g., by welding) to a proximal end of tube 110.

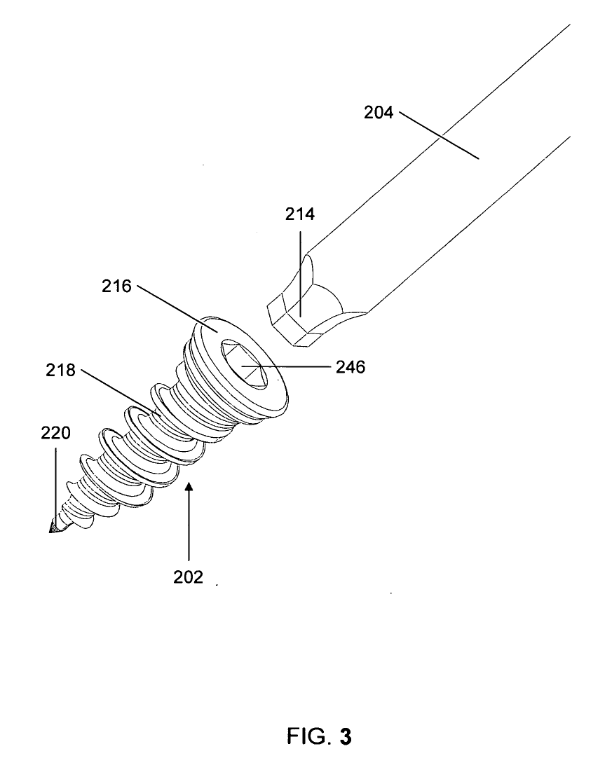

FIG. 3 is a perspective view of another embodiment of a bone anchor 202 and a rod 204 according to the present invention. In this embodiment, rod 204 consists essentially of a single, elongate shaft (similar to shaft 112 (FIG. 2)), which detachably couples to bone anchor 202 and causes anchor 202 to advance into bone, for example, upon impact or rotation of rod 204. Rod 204 may be coupled (e.g., by welding) at its proximal end to, for example, a handle such as handle 130 (FIGS. 1 and 4). As in the embodiment of FIG. 1, bone anchor 202 is a bone screw that includes screw head 216 and threaded shaft 218 ending in tip 220 distal from screw head 216. Screw head 216 contains socket 246, which may be a hex socket having six sides of equal lengths. In other embodiments, socket 246 can have other cross-sectional shapes including, but not limited to, slotted, Phillips, hexalobe and Robertson. Tapered end 214 has a cross-sectional area that is complementary to the cross-sectional area of socket 246. An interference fit is formed upon inserting tapered end 214 of rod 204 into socket 246 of screw head 216.

FIG. 4 is an enlarged, cross-sectional view of handle 130 and a proximal portion of rod 104 of the embodiment of FIG. 1. In some embodiments, handle 130, which is rigidly attached (e.g., by welding) to a proximal portion of tube 110, includes a spring 170 and a plunger 132 that push knob 122 and/or shaft 112 towards screw 102, thereby securing shaft 112 within tube 110 and permitting alignment of distal end 114 of shaft 112 with a complimentary feature in anchor head 116. The spring plunger mechanism is located within cylinder 164 in through bore 166 that runs through and is coaxial with handle 130. At the end of cylinder 164 near knob 122 and shaft 112, bore 168 is formed partway through and is coaxial with cylinder 164. Spring 170 is compressed within bore 168 proximal to knob 122 and shaft 112. Between spring 170 and knob 122/shaft 112 is plunger 132. The spring force from spring 170 transmitted through plunger 132 to knob 122 and/or shaft 112 secures shaft 112 within tube 110. Cylinder 164 is retained within handle 130 by toroidal spring or o-ring 172 which snaps into internal groove 174 within handle 130 and cylinder 164. To permit cleaning, replacement and/or repairs, cylinder 164 can be removed from handle 130, thereby allowing the removal of the spring plunger mechanism and shaft 112.

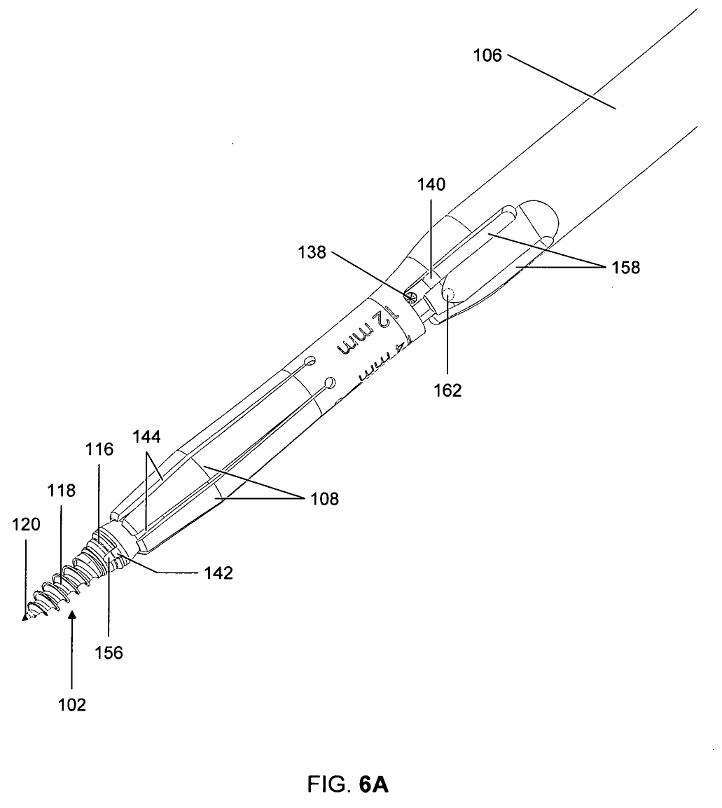

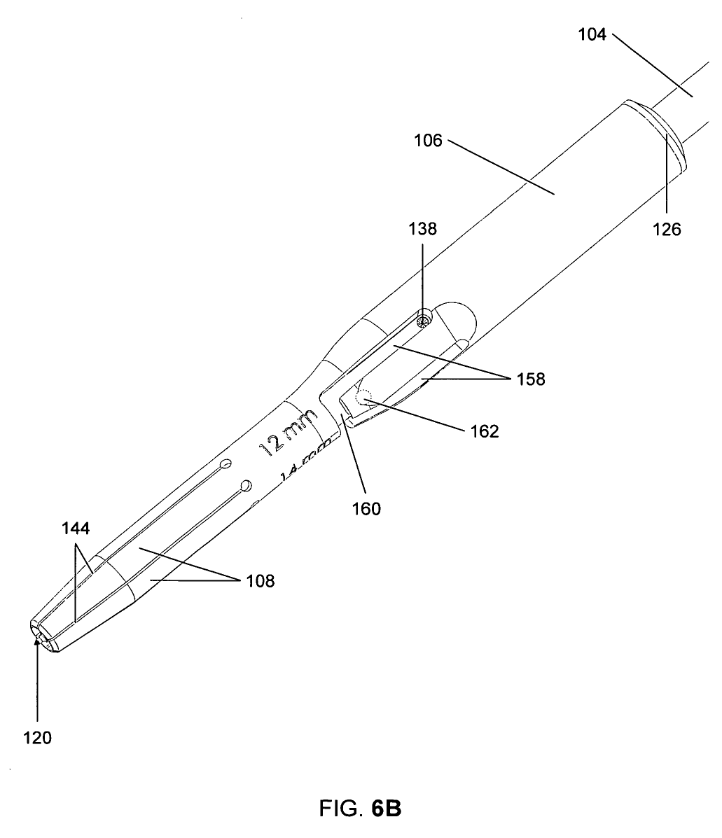

FIG. 5A is an enlarged, cross-sectional, perspective view of a distal portion of the tool of assembly 100 of the embodiment of FIG. 1, in which housing member 106 (including flexible fingers 108) is in a proximal position relative to rod 104 (including tube 110 and shaft 112) and rod 104 is ready for engagement with a bone anchor. In some embodiments, flexible fingers 108 traverse the entire circumference of a circle transverse to a longitudinal axis of housing member 106. For example, N equally sized flexible fingers 108 can be formed by cutting at the distal end of housing member 106 N narrow slots 144 (FIGS. 1 and 6A) along the longitudinal axis and at equidistant positions around the entire perimeter of housing member 106. In other embodiments, flexible fingers 108 only traverse part of the circumference of a circle transverse to a longitudinal axis of housing member 106.

FIG. 5B shows a distal portion of assembly 100 of FIG. 1, in which the housing member including flexible portion 108 is in a distal position relative to tube 110 and shaft 112 of rod 104, with bone anchor 102 loaded within the housing member and prepared for insertion into bone. In some embodiments, in this position flexible portion 108 may house most or all of anchor 102 except for, for example, tip 120. The inner diameter of the flexible portion 108 may be smallest (e.g., tapered or having a step) near its distal end. When anchor 102 is within housing member 106, some or all of the flexible portion of housing member 106 may be configured to contact at least some of the threads of threaded shaft 118 and/or tip 120.

Returning to FIG. 5A, following coupling of anchor 102 to rod 104, the surgeon slides housing member 106 distally along an axis of rod 104 in the direction towards anchor 102. Since the distal end of tube 110 may be at least as wide as anchor head 116, flexible fingers 108 flex outward to allow passage of anchor head 116 as it enters housing member 106. Once anchor head 116 has cleared the narrowest region in the distal end of housing member 106, flexible fingers 108 begin to relax. When tip 120 of anchor 102 is generally aligned with (e.g., slightly protruding) from the distal end of housing member 106, flexible fingers 108 may snugly or closely fit at least a portion of threaded shaft 118 by contacting some of the threads of threaded shaft 118. In the resulting configuration, as shown in FIG. 5B, assembly 100 is prepared for the insertion of screw 102 into a bone. In some embodiments, the distal end of housing member 106 is configured to fit within or otherwise align with a bore in a bone plate, such as a spinal fixation plate (e.g., a cervical bone plate). For example, the distal end of the flexible portion 108 may have a geometry that is complimentary to a configuration of the bore of the bone plate, thereby allowing the surgeon to center anchor 102 within the bore by resting the distal end of housing member 106 within the bore. As anchor 102 is advanced out of housing member 106 and into the bone, portion 108 again flexes outwardly from a longitudinal axis of housing member 106 to allow passage of anchor 102.

Tip 120 of anchor 102 may have a sharp point (e.g., a conical point) that can form a hole in a bone surface when impacted or otherwise seated into a bone. Bone anchor 102 and/or the other components of assembly 100 may be formed from any suitable material or combination of materials such as, for example, biocompatible metal (e.g., titanium) or ceramic. For example, in some embodiments, bone anchor 102 may include the high strength, high toughness doped silicon nitride ceramic described in commonly-owned U.S. Pat. No. 6,881,229, which is hereby incorporated by reference herein in its entirety. Therefore, acting as an awl, tip 120 of anchor 102 can be seated in a bone by impacting assembly 100. For example, the surgeon can impact the flat, rigid surface provided by handle top 134 with a hammer or mallet to cause anchor tip 120 to penetrate the cortical bone of a patient. Alternatively, the surgeon may seat tip 120 into bone by applying a more continuous force to and/or twisting assembly 100. In some embodiments, the threads on threaded shaft 118 are self-drilling and/or self-tapping. Once tip 120 has been seated into a bone, the surgeon can advance bone screw 102 into the bone with a push and turn motion on handle 130.

Returning to FIGS. 1 and 2, in some embodiments, tube 110 has a tapered section and/or recessed section 136 near its distal end. Recessed section 136 is configured so that when housing member 106 is retracted proximally relative to tube 110 (FIGS. 1 and 5A), flexible fingers 108 can nest within recessed section 136. This allows the surgeon to expose rod 104 for coupling to anchor 102 at the start of the surgical procedure and provides for retraction of housing member 106 for improved visualization of anchor 102 during the surgical procedure.

Referring to FIGS. 6A and 6B, an indicator may be provided that indicates the position of rod 104, and the bone anchor 102 coupled to its distal end, relative to housing member 106 and that allows the surgeon to properly distally position housing member 106 based on the length of the screw. Housing member 106 has one or more slots 158 running longitudinally along the middle of housing member 106. In some embodiments, multiple slots 158 may be provided, one for each of a plurality of screw lengths. For example, there may be three slots 158 for 12 mm, 14 mm and 16 mm screws, respectively. In embodiments with more than one slot 158, a circumferential slot 160 may be provided that is cut circumferentially around part of housing member 106 connecting slots 158 at the ends closest to the flexible portion (e.g., fingers 108) of housing member 106. On tube 110, an indicator (e.g., bright dot) 138 may be provided that is visible through slots 158 of housing member 106 to indicate position. Alternatively, a guide pin that slides along slots 158 can be used instead of or in addition to indicator 138. As shown in FIG. 6A, with housing member 106 fully retracted, dot 138 is visible through the distal end of slot 158. As housing member 106 is slid distally, dot 138 traverses longitudinally along slot 158 away from the flexible portion of housing member 106. When housing member 106 is fully advanced and encompasses the desired screw length (e.g., 12 mm), as shown in FIG. 6B, dot 138 is visible through the proximal end of slot 158.

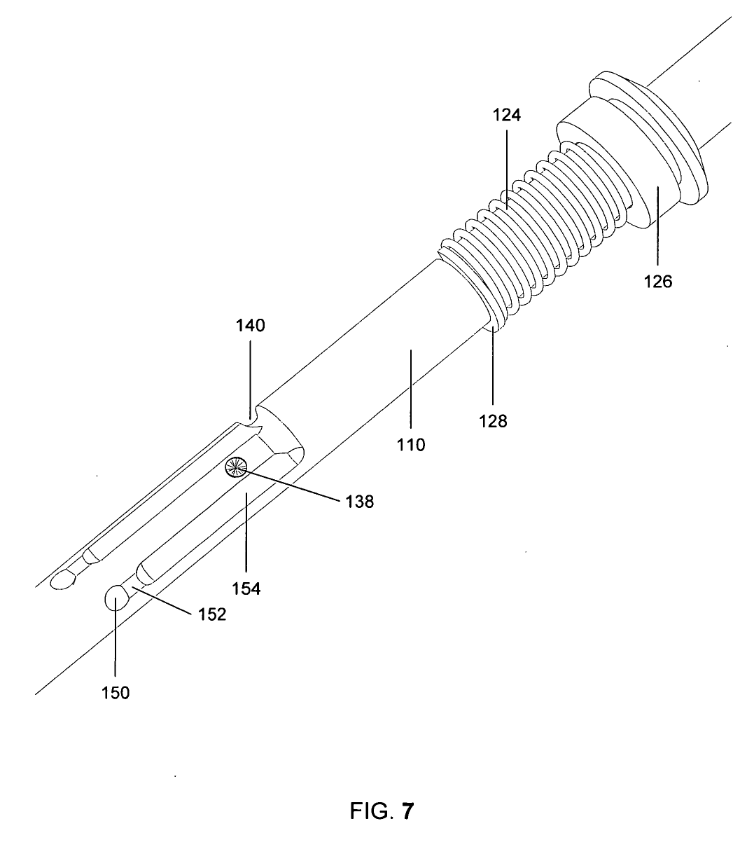

FIG. 7 is an enlarged, perspective view of part of bone anchor insertion assembly 100 of FIG. 1 showing the features for facilitating positioning and/or movement of the housing member relative to the rod, according to some embodiments of the present invention. Referring to FIGS. 2 and 7, a mechanism is provided that includes compression spring 124, cap 126 and C-clip 128. C-clip 128 is attached to tube 110 and retains compression spring 124 on tube 110. Cap 126 is located along tube 110 and abuts (e.g., with or without rigidly connecting to) the end of compression spring 124 opposite C-clip 128. Cap 126 is attached to the proximal end of housing member 106, for example, by threading into housing member 106. Cap 126 can alternatively be attached to housing member 106 in other ways known in the art, including but not limited to press fitting, attachment via one or more screws or by gluing or welding. Following coupling of anchor 102 to rod 104, as shown in FIG. 6A, the surgeon can slide the housing member 106 distally along rod 104 and/or pull rod 104 in the proximal direction, thereby compressing compression spring 124 until tip 120 is generally aligned with (e.g., slightly protruding from) the distal end of housing member 106, as shown in FIG. 6B.

The mechanism according to the embodiment shown in FIG. 7 also facilitates retraction of housing member 106 proximally along rod 104, thereby allowing for visualization of the surgical procedure by the surgeon. The retraction can be automatic or may be performed manually by the surgeon. In some embodiments, the mechanism automatically releases and causes housing member 106 to retract once anchor 102 has advanced a predetermined distance into bone, for example, a distance sufficient to define the trajectory of anchor 102 upon further advancement in the absence of guidance from housing member 106. Such an automatic release mechanism can indicate to the surgeon when to stop impacting handle top 134 and to start rotating handle 130. It can also prevent the flexible portion 108 of housing member 106 from binding within the bore of a bone plate as flexible portion 108 expands.

In some embodiments, tube 110 of rod 104 includes recess (e.g., dimple) 150, elevated groove 152, and sliding groove 154 configured for receipt of ball detent 162 (FIGS. 6A and 6B) located on an inner surface of housing member 106 (e.g., on a tongue formed by two slots 158 and circumferential slot 160). Grooves 152 and 154 may be parallel to a longitudinal axis of tube 110. When ball detent 162 is positioned within recess 150, rod 104 and housing member 106 may be fixed in a temporarily fixed position, such as the configuration shown in FIG. 6B in which the distal end of housing member 106 is ready for coupling to a bone fixation plate and anchor tip 120 is generally aligned with the distal end of housing member 106.

Upon impact of handle top 134 (FIG. 1) when the distal end of housing member is seated within a bone plate, anchor tip 120 may advance into the bone and ball detent 162 may eject from recess 150 and into groove 152, corresponding to a slight distal-to-proximal movement of housing member 106 relative to tube 110 of rod 104. Groove 152 may be elevated relative to both recess 150 and sliding groove 154, which may create sufficient resistance between groove 152 and ball detent 162 to temporarily fix the relative positions of tube 110 and housing member 106. Upon continued impact of handle top 134, ball detent 162 may advance (e.g., stepwise with each tap of handle top 134) along elevated groove 152 for its length corresponding to further distal-to-proximal movement of housing member 106 relative to rod 104. Once ball detent 162 has advanced the entire length of elevated groove 152 (e.g., 1-2 mm), it may connect with sliding groove 154 through which ball detent 162 may slide freely and automatically in the distal-to-proximal direction under the spring force stored in compression spring 124. The length of elevated groove 152 may correspond generally to the depth of anchor 102 within the bone at which further guidance by housing member 106 is not needed. Ball detent 162 may cease sliding once it reaches the end of groove 154. This proximal position of housing member 106 is shown in FIG. 6A. At this point, tip 120 is seated in the bone and the surgeon may advance anchor 102 further into the bone by rotating handle 130 coupled to rod 104.

In some embodiments, tube 110 of rod 104 may include multiple sets of recesses 150, elevated grooves 152, and sliding grooves 154, for example, one set for each desired length of anchor 102 (e.g., 12 mm, 14 mm and 16 mm screws). Circumferential groove 140 may be provided that extends around at least part of the circumference of tube 110 connecting the sliding grooves 154 at their proximal ends. With ball detent 162 positioned within circumferential groove 140, the housing member 106 can be rotated around the longitudinal axis of rod 104 to align ball detent 162 with the particular sliding groove 154 corresponding to a desired anchor length. In some embodiments, proper alignment can be achieved by aligning indicator 138 on tube 110 with a marking (e.g., “12 mm”) corresponding to a desired anchor length formed on housing member 106, as shown in FIG. 6A. In some embodiments, ball detent 162 can be derailed out of the channels formed in tube 110 to allow for disassembly of housing member 106 from rod 104 for cleaning and/or adjustments.

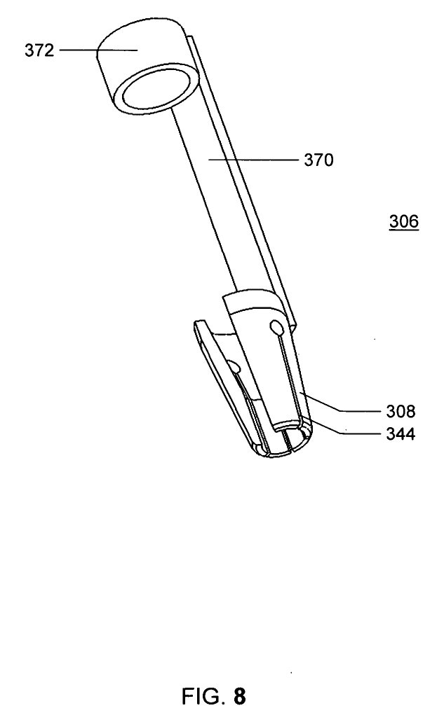

FIG. 8 is an enlarged, perspective view of another embodiment of an elongated housing member 306 according to the present invention. At the distal or screw receiving end of housing member 306 is a flexible portion capable of flexing in a direction away from a longitudinal axis of housing member 306. In some embodiments, the flexible portion of housing member 306 includes a plurality (e.g., five) flexible members, for example, flexible fingers 308. For example, flexible fingers 308, can be formed by cutting narrow slots 344 at the distal end of housing member 306. Slots 344 may be generally aligned with the longitudinal axis of member 306 and may extend around at least part of the perimeter of member 306. In this example, housing member 306 only traverses part of the circumference of a circle transverse to its longitudinal axis, although in other embodiments housing member 306 may traverse the entire circumference of a circle transverse to its longitudinal axis. Strut 370 may be provided that connects the flexible portion of member 306 to portion 372 (e.g., a ring) that couples to the rod (e.g., rod 104 (FIG. 2)). A surface at the distal end of strut 370 may be rigidly attached to (e.g., by welding) or integrally formed with a proximal end of housing member 306. A long axis of strut 370 may be parallel to the longitudinal axis of housing member 306, and the proximal end of strut 370 may be rigidly attached to or integrally formed with the outside of ring 372.

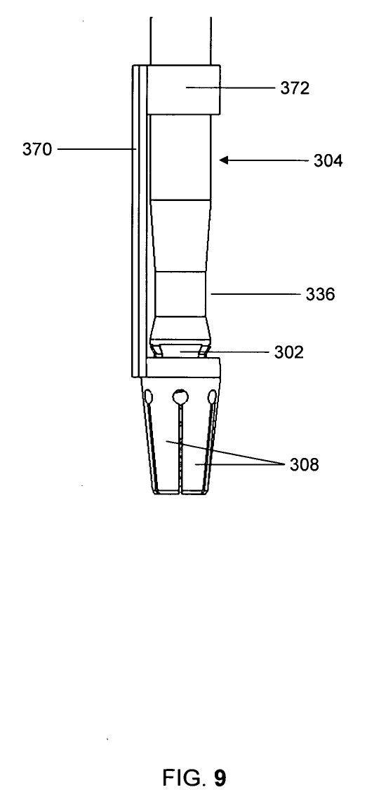

FIG. 9 is an enlarged, side view of the housing member 306 of FIG. 8 incorporated within a bone anchor insertion assembly according to some embodiments of the present invention. As shown, the proximal end of a bone anchor 302 may be at least partially visible to a surgeon when housing member 306 is in a distal position relative to rod 304. Rod 304 may be the same as or similar to rod 104 (FIG. 1) or rod 204 (FIG. 3). Similarly, bone anchor 302 may be the same as or similar to bone screw 102 (FIG. 1) or bone anchor 202 (FIG. 3). Rod 304 runs longitudinally through and can translate axially relative to ring 372, with rod 304 being centered relative to the longitudinal axis of housing member 306. Anchor 302 is capable of being detachably coupled to rod 304. When bone anchor 302 is within housing member 306, at least a portion of the anchor may contact at least part of the flexible portion of housing member 306. Recessed section 336 of rod 304 may be configured to receive flexible fingers 308 when housing member 306 is in a proximal position relative to rod 304.

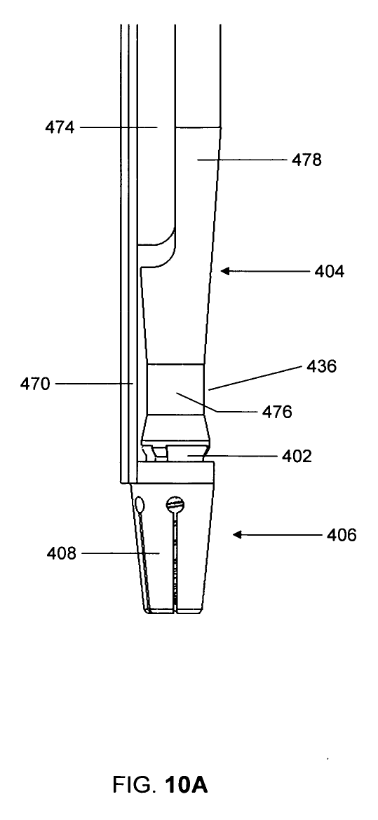

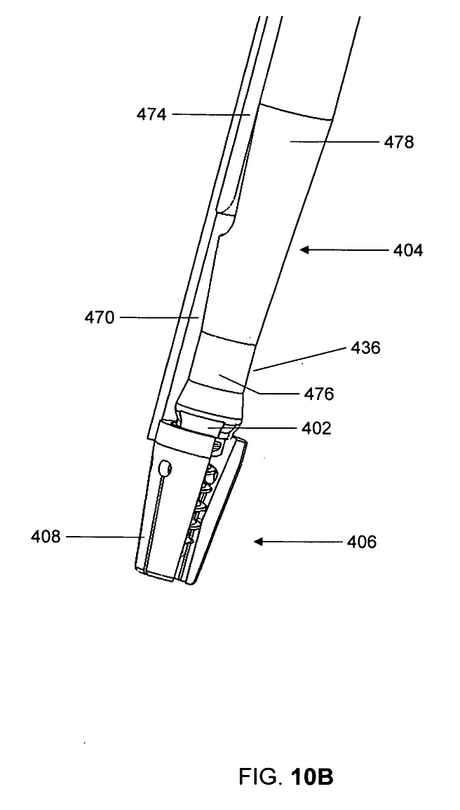

FIGS. 10A and 10B are enlarged, side and perspective views, respectively, of another bone anchor insertion assembly according to some embodiments of the present invention, in which a housing member is provided that is configured for slidable engagement with a rod. Housing member 406, which includes strut 470 and flexible portion 408, may be the same or similar to housing member 306 (FIGS. 8 and 9) in all respects, except that member 406 may include portion 474 that includes a channel, tab, or rail configured for slidable engagement with a complimentary tab, rail, or channel in rod 404. Rod 404 includes recessed section 436 for receiving flexible portion 408 of housing member 406. In some embodiments, rod 404 includes lower portion 476 and upper portion 478. When lower portion 476 is coupled to bone anchor 402, portion 476 has a longitudinal axis that is coaxial with the longitudinal axis of bone anchor 402 and a longitudinal axis of housing member 406. Upper portion 478 of rod 404 has a longitudinal axis that is offset relative to the longitudinal axis of lower portion 476. The features in lower portion 476 of rod 404 for coupling to bone anchor 402 may be the same as or similar to the corresponding coupling features shown and described in connection with rod 104 (FIG. 1), rod 204 (FIG. 3), or rod 304 (FIG. 9). Advancement of rod 404 (e.g., upon impact or rotation) results in advancement of bone anchor 402.

Thus it is seen that assemblies and tools, as well as methods for their use, are provided for inserting a bone screw into a bone without having to create a pilot hole. Although particular embodiments have been disclosed herein in detail, this has been done by way of example for purposes of illustration only, and is not intended to be limiting with respect to the scope of the appended claims, which follow. In particular, it is contemplated that various substitutions, alterations, and modifications may be made without departing from the spirit and scope of the invention as defined by the claims. Other aspects, advantages, and modifications are considered to be within the scope of the following claims. The claims presented are representative of the inventions disclosed herein. Other, unclaimed inventions are also contemplated. The applicant reserves the right to pursue such inventions in later claims.

What is claimed is:

1. A bone anchor insertion apparatus comprising:
a housing member having a distal end and a proximal end, wherein said housing member is configured to receive a bone anchor having a proximal end and a tip at its distal end such that said tip of said bone anchor is generally aligned with said distal end of said housing member with the rest of said bone anchor being positioned at least partially within said housing member; and
a rod coupled to said housing member, wherein said rod is configured for detachably coupling to said proximal end of said bone anchor such that advancement of said rod causes advancement of said bone anchor.

2. The apparatus of claim 1 wherein said housing member is configured to receive said bone anchor through said distal end of said housing member.

3. The apparatus of claim 1 wherein said distal end of said housing member is configured for positioning within a bore of a bone plate such that said tip of said bone anchor is centered within said bore when said bone anchor is coupled to said rod and said tip of said bone anchor is generally aligned with said distal end of said housing member.

4. The apparatus of claim 1, wherein said housing member is configured such that said tip of said bone screw protrudes from said distal end of said housing member.

5. The apparatus of claim 1, wherein said housing member is configured such that said bone screw protrudes about 1 to 2 millimeters from said distal end of said housing member.

6. The apparatus of claim 1, wherein said housing member is configured such that said tip of said bone screw is generally flush with said distal end of said housing member.

7. The apparatus of claim 1, wherein said housing member is configured such that said tip of said bone screw is recessed within said distal end of said housing member.

8. The apparatus of claim 1, wherein the rest of said bone screw is positioned entirely within said housing member when said tip of said bone screw is generally aligned with said distal end of said housing member.

9. The apparatus of claim 1, wherein said rod is configured for positioning at least partially within said housing member.

10. The apparatus of claim 1, wherein said rod and said housing member are configured to move relative to each other from a proximally advanced position of said housing member relative to said rod to a distally advanced position of said housing member relative to said rod, wherein in said distally advanced position said tip of said bone anchor is generally aligned with said distal end of said housing member.

11. The apparatus of claim 10, further comprising a retraction mechanism for retracting said housing member in a distal-to-proximal direction relative to said rod.

12. The apparatus of claim 11, wherein said retraction mechanism comprises a spring positioned in a compressed state when said apparatus is in said distally advanced position of said housing member relative to said rod.

13. The apparatus of claim 12, wherein said spring is in a relaxed state when said apparatus is in said proximally advanced position of said housing member relative to said rod.

14. The apparatus of claim 11, further comprising a feature formed in said housing member and a complimentary feature formed in said rod for holding said housing member temporarily fixed relative to said rod when said when said apparatus is in said distally advanced position of said housing member relative to said rod.

15. The apparatus of claim 14, wherein said feature formed in said housing member comprises a ball detent.

16. The apparatus of claim 1, wherein said rod has a longitudinal axis along which said housing member is configured to move relative to said rod.

17. The apparatus of claim 1, wherein said rod comprises a tube and a shaft extending at least partially through said tube, wherein each of said tube and said shaft are configured for detachable coupling to said proximal end of said bone anchor.

18. The apparatus of claim 17, wherein said shaft is threaded at the distal end for mating with a threaded bore within said proximal end of said anchor.

19. The apparatus of claim 1, wherein said rod comprises a plurality of prongs configured for mating with complimentary recesses in said proximal end of said bone anchor.

20. The apparatus of claim 1, wherein said rod is coupled to a handle.

21. The apparatus of claim 1, wherein said housing member comprises an elongated sleeve.

22. The apparatus of claim 21, wherein said sleeve has a circular cross-section.

23. The apparatus of claim 1, wherein said distal end of said housing member comprises a flexible portion capable of flexing in a direction away from an axis of said bone anchor.

24. The apparatus of claim 23, wherein said flexible portion comprises one or more flexible fingers.

25. The apparatus of claim 1 further comprising an indicator for indicating the position of said rod relative to said housing member.

26. The apparatus of claim 1 further comprising said bone anchor.

27. The apparatus of claim 26, wherein the tip of said bone anchor forms a sharp point capable of forming a hole in a bone surface.

28. The apparatus of claim 26, wherein said bone anchor comprises a bone screw and wherein a diameter of a head of said screw is larger than the diameters of the threads on a threaded shaft of said screw.

29. A bone anchor insertion apparatus comprising:
a housing member having a distal end and a proximal end, wherein said distal end of said housing member is configured for receipt within a bore of a bone fixation plate; and
a rod coupled to said housing member, wherein a distal end of said rod is configured for detachably coupling to a proximal end of a bone anchor such that rotation of said rod causes rotation of said bone anchor;
wherein said rod and said housing member are configured for movement relative to each other between a proximally advanced position of said housing member relative to said rod and a distally advanced position of said housing member relative to said rod;
wherein in said proximally advanced position said distal end of said rod is distal to said distal end of said housing member; and
wherein in said distally advanced position said distal end of said rod is proximal to said distal end of said housing member.

30. The apparatus of claim 29 further comprising said bone anchor.

31. The apparatus of claim 30 further comprising said bone fixation plate.

32. Apparatus for inserting a bone anchor into a bone comprising:
first means for guiding insertion of a bone anchor into bone;
second means, coupled to said first means, for advancing said bone anchor into a bone in response to an impact;
means for sliding said first means proximally to said second means; and
means for further advancing said bone anchor into said bone in response to a rotational force.

33. A method for inserting a bone anchor into a bone comprising:
positioning a bone anchor having a proximal end and a tip at its distal end within a housing member such that said tip is generally aligned with a distal end of the housing member;
coupling said distal end of said housing member to a bore of a bone fixation plate;
advancing said bone anchor into a bone by impacting a proximal end of a rod coupled to said proximal end of said bone anchor and to said housing member;
sliding said housing member proximally relative to said rod; and
further advancing said bone anchor into said bone upon rotation of said rod.

34. The method of claim 33 wherein said sliding occurs automatically once said tip of said anchor is seated in said bone.

35. The method of claim 33 wherein said advancing said bone anchor into a bone comprises advancing said bone anchor into a bone without creating a pilot hole.
